(19) United States
(12) Patent Application Publication
(10) Pub. No.: US 2017/0143740 A1
(43) Pub. Date: May 25, 2017

(54) ANESTHETIC FORMULATION

(71) Applicant: Ramon Cabreja, Coral Gables, FL (US)

(72) Inventor: Ramon Cabreja, Coral Gables, FL (US)

(21) Appl. No.: 14/950,941

(22) Filed: Nov. 24, 2015

(51) Int. Cl. A61K 31/573; A61K 31/445; A61K 9/00; A61K 31/137

(52) U.S. Cl. CPC A61K 31/573; A61K 31/137; A61K 31/445; A61K 9/0019

(57) ABSTRACT

The present invention relates to compositions and methods for pain reduction, including but not limited to, peripheral nerve blocks.

### FIELD OF THE INVENTION

The present invention relates to compositions and methods use in pain reduction, including but not limited to, peripheral nerve blocks. In particular, the present invention relates to compositions and methods for the administration of a peripheral nerve block formulation comprising of bupivacaine or ropivacaine in combination with dexamethasone and epinephrine.

All documents cited to or relied upon below are expressly incorporated herein by reference.

### BACKGROUND OF THE INVENTION

The prevention of pain is a critical component of medical practice, whether it is needed for the alleviation of endemic pain due to disease, injury, or other conditions, or to enable medical or surgical procedures.

Peripheral nerve blocks are used frequently in a variety of surgical procedures for surgical anesthesia and postoperative pain. Long-acting local anesthetics alone can provide analgesia for 9-14 hours (Casati et al. (2000) Minerva Anesesiol. 66:39-44; Hickey et al. (1991) Anesthesiol 74:639-642; Hickey et al. (1992) Anesth. Analg. 75:602-606; Vaghadia et al. (1999) Can J. Anaesth. 46:946-951). If the block is performed in the morning or early afternoon, patients commonly report postoperative pain during nighttime hours. The need for opioids leads to the potential for opioid-induced side effects, including the inhibition of restorative sleep (Lydic et al., in Sleep and Pain, ed. by G. Lavigne, B J Sessle, M Choinire M, P J Soja, (2007) IASP Press, Seattle, Wash. pp. 99-122) and the potential for airway obstruction and desaturation (Bonafide et al. (2008) Anesthesiol. 108:627-633; Bowdle (2004) Anesth. Analg. 99:70-76; Rosenberg et al. (1995) Eur. J. Anaesthesiol. Suppl. 10:28-30). Ideally, single shot peripheral nerve blocks would provide analgesia throughout the first postoperative night.

Many additives to local anesthetics have been investigated in an attempt to increase the duration of the block in order to improve postoperative pain. The efficacy of clonidine, an α₂-adrenoceptor agonist, has been established in a variety of regional anesthesia techniques (Eisenach et al. (1996) Anesthesiol. 85:655-674). Clonidine has been combined with dexamethasone, epinephrine and bupivacaine in a four-drug cocktail in an ultrasound-guided interscalene brachial plexus blockade. (Vieira et al., (2010) Eur. Soc. Anaesthesiology, 27(3): 285-288). Clonidine has been shown in clinical studies to prolong the duration of anesthesia and analgesia in peripheral nerve blocks, although results with long acting local anesthetics have been unsuitable (Cucchiaro et al. (2007) Anesth. Analg. 104:532-537; Casati et al. (2000) Anesth. Analg. 91:388-92; El Saied et al. (2000) Can. J. Anaesth. 47:962-967; Eledjam et al. (1991) Can. J. Anaesth. 38:870-875; Hutschala et al. (2004) Eur. J. Anaesthesiol. 21:198-204; Iohom et al. (2005) Anesth. Analg. 100:1179-1183; Iskandar et al. (2003) Anesth. Analg. 96:260-262; Iskandar et al. (2001) Anesth. Analg. 93:771-775; Reinhart et al. (1996) Anesth. Analg. 83:760-765; Singelyn et al. (1992) Reg. Anesth. 17:148-150; Singelyn et al. (1996) 83:1046-1050). Some studies have found no beneficial effect with the addition of clonidine (Culebras et al. (2001) Anesth. Analg. 92:199-204; Gaumann et al. (1992) Anesth. Analg. 75:69-74; Erlacher et al. (2000) Acta Anaesthesiol. Scand. 44:53-57).

Although ropivacaine and bupivacaine are both long-acting local anesthetics, ropivacaine has unique pharmacologic properties and has replaced bupivacaine for peripheral nerve blocks in many institutions in the United States. The predominate reason for the change is the belief that ropivacaine is more likely to respond to resuscitation efforts in the event of cardiac arrest from intravascular injection when compared with bupivacaine (Chazalon et al. (2003) anesthesiol. 99:1449-1451; Khoo et al. (2006) Anaesth. Intensive Care 34:804-807; Klein et al. (2003) Anesth. Analg. 97:901-903; Polley et al. (2003) Anesthesiol. 99:1253-1254; Ohmura et al. (2001) Anesth. Analg. 93:743-748; Litz et al. (2006) Anesthesia 61:800-801; Hansen (2004) Expert Rev. Neurother. 4:781-791; Simpson et al. (2005) Drugs 65:2675-2717). In addition to a safer cardiac profile, some studies have shown that ropivacaine is associated with less motor blockade when compared with bupivacaine (Hansen (2004) Expert Rev. Neurother. 4:781-791; Simpson et al. (2005) Drugs 65:2675-2717; Dyhre et al. 1997) Acta Anaesthiol. Scand. 41:1346-1352; Zink et al. (2004) Drug Saf. 27:1093-1114). Improved motor function while maintaining analgesia allows patients to participate in physical therapy and improves postoperative function. Selectivity for C and A-delta fibers compared with A-alpha fibers has been demonstrated with clonidine (Butterworth et al. (1993) Anesth. Analg. 76:295-301; Gaumann et al. (1992) Anesth. Analg. 74:719-725).

Peripheral nerve blocks may be used for nerves outside of the central nervous system, and find particular use in outpatient surgical settings. However, peripheral nerve blocks pose particular problems in terms of safety and effective duration; that is, drugs and dosages that are found to be effective and safe when applied to nerves of the central nervous system (e.g., during epidural injection) do not predictably have the same systemic toxicity, neurotoxicity, and effectiveness when applied to peripheral nerve blocks.

Therefore, there is a need for safer, longer-lasting, and more effective anesthetic and analgesic compositions and methods for peripheral nerve blocks.

### SUMMARY OF THE INVENTION

Provided is an anesthetic formulation, comprising a therapeutically effective amount of each of dexamethasone, epinephrine and bupivacaine or ropivacaine, or a pharmaceutically acceptable salt thereof, and a pharmaceutically acceptable carrier. Also provided is a method for reducing pain in a subject in need thereof, comprising the step of administering a therapeutically effective amount of each of dexamethasone, epinephrine and bupivacaine or ropivacaine, or a pharmaceutically acceptable salt thereof, as a peripheral nerve block.

### DETAILED DESCRIPTION OF THE INVENTION

It is to be understood that the descriptions of the present invention have been simplified to illustrate elements that are relevant for a clear understanding of the present invention, while eliminating, for the purpose of clarity, many other elements found in typical pharmaceutical compositions. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

The present invention relates to compositions and methods for reducing pain, including but not limited to, methods and compositions for peripheral nerve blocks. In particular, the present invention relates to the administration of dexamethasone, epinephrine and bupivacaine or ropivacaine in peripheral nerve blocks. In one embodiment of the invention, dexamethasone, epinephrine and bupivacaine or ropivacaine, or pharmaceutically acceptable salts thereof, are the only analgesic agents in the composition.

In certain embodiments, methods and compositions of the present invention relates to regional or peripheral nerve blocks. Types of regional or peripheral nerve blocks include but are not limited to motor nerve blocks, sensory nerve blocks, differential nerve blocks, autonomic nerve blocks, brachial plexus nerve blocks (axillary, interscalene, supraclavicular, infraclavicular), individual upper extremity nerve blocks (median, radial, ulnar, musculocutaneous, axillary), sciatic nerve blocks, ankle nerve blocks, metatarsal nerve blocks, oral nerve blocks, femoral nerve blocks, popliteal fossa nerve blocks, saphenous nerve blocks, distal nerve blocks, digital nerve blocks, deep peroneal nerve blocks, superficial peroneal nerve blocks, tibial nerve blocks, sural nerve blocks, and saphenous nerve blocks.

In certain embodiments, the invention provides methods and compositions for reducing pain, comprising administering dexamethasone and epinephrine in combination with a long-acting local anesthetic such as bupivacaine or ropivacaine.

Methods and compositions of the present invention are not limited by the route of delivery or routes of administration by which therapeutic agents are administered. Routes of delivery include but are not limited to single injection, serial injections, indwelling catheter and continuous infusion. Routes of administration include but are not limited to subcutaneous, intraperitoneal, oral or intrapulmonary.

The methods and compositions of certain embodiments of the present invention find use in treating many types of pain. Methods and compositions of certain embodiments of the present invention may be used to treat existing pain (e.g. pain associated with an injury, previously performed medical procedure, or pathological condition), or for the prevention of pain (e.g., prior to or during a surgical or other medical procedure). Types of pain include but are not limited to acute pain, chronic pain, thermal pain, traumatic pain, chemical pain, inflammatory pain, ischemic pain, blunt pain, sharp pain, prickling pain, visceral pain, and neuropathic pain.

Furthermore, methods and compositions of certain embodiments of the present invention find use in treating subjects of various types. Methods and compositions of the present invention may be used in a variety of settings including but not limited to human medical (e.g., clinical, emergency, search-and-rescue), veterinary (e.g., for companion animal subjects, for livestock subjects, for wildlife subjects), or research (e.g., for research animals). Subjects include but are not limited to dogs, cats, rats, mice, rabbits, horses, bovines, goats, pigs, primates, and humans. In preferred embodiments, the subject is a human. The human subject may have an underlying or additional condition. Such conditions may include but are not limited to advanced age, neonatal status, pediatric status, impaired pulmonary function, impaired liver function, impaired cardiovascular function, impaired metabolic function, impaired renal function, impaired gastrointestinal function, impaired neuromuscular function, pregnancy, diabetes, impaired cognitive function, and impaired tolerance of anesthetic and/or analgesic agents (e.g., due to genetic predisposition or metabolic condition).

In certain embodiments, the present invention provides kits for use with methods or compositions for reduction of pain in a subject. In preferred embodiments, the subject is a human. In some embodiments, the kit provides, a composition for reduction of pain. In some embodiments, the kit further comprises components that include but are not limited to compositions described herein, syringes, stimulating needles, antiseptic wipes or solutions, sedative agent(s), labels, written and/or pictorial instructions and product information, and package environmental controls (e.g., ice, desiccants, etc).

In certain embodiments, the present invention provides a method for reducing pain in a subject, comprising administering dexamethasone and epinephrine in combination with a long-acting local anesthetic as a peripheral nerve block. In some embodiments, the long-acting local anesthetic is an agent such as bupivacaine and ropivacaine.

In some embodiments, the long-acting local anesthetic is ropivacaine. In some embodiments, the dose of dexamethasone is 0.18 to 0.25 mg/cc. For example, the dose of dexamethasone is 0.18 mg/cc, 0.19 mg/cc, 0.20 mg/cc, 0.21 mg/cc, 0.22 mg/cc, 0.23 mg/cc, 0.24 mg/cc or 0.25 mg/cc.

In some embodiments, the dose of epinephrine is 1:150,000 to 1:250,000. In certain embodiments the dose of bupivacaine or ropivacaine is 0.30 to 0.35% total volume of the formulation. For example, the dose of bupivacaine or ropivacaine is 0.30, 0.31, 0.32, 0.33, 0.34 or 0.35% total volume of the formulation.

In some embodiments, the subject is a human. In some embodiments, the human subject has an additional condition such as advanced age, neonatal status, pediatric status, impaired pulmonary function, impaired liver function, impaired cardiovascular function, impaired metabolic function, impaired renal function, impaired gastrointestinal function, impaired neuromuscular function, pregnancy, diabetes, impaired cognitive function, and impaired tolerance of anesthetic or analgesic agents.

In some embodiments, the peripheral nerve block is a type such as a motor nerve block, a sensory nerve block, a differential nerve block, an autonomic nerve block, a brachial plexus nerve block (axillary, interscalene, supraclavicular, infraclavicular), an individual upper extremity nerve block (median, radial, ulnar, musculocutaneous, axillary), a sciatic nerve block, ankle nerve block, metatarsal nerve block, oral nerve block, femoral nerve block, popliteal fossa nerve block, saphenous nerve block, distal nerve block, digital nerve block, deep peroneal nerve block, superficial peroneal nerve block, tibial nerve block, sural nerve block, or saphenous nerve block. In some embodiments, more than one peripheral nerve block is performed. In some embodiments, the delivery route may be a single injection, serial injections, indwelling catheter and continuous infusion. In some embodiments, the route of administration is a type such as subcutaneous, intraperitoneal, oral, intrapulmonary and/or intramuscular. In some embodiments, the type of pain includes but is not limited to acute pain, chronic pain, thermal pain, traumatic pain, chemical pain, inflammatory pain, ischemic pain, blunt pain, sharp pain, prickling pain, visceral pain, and/or neuropathic pain.

Additional embodiments will be apparent to persons skilled in the relevant art based on the teachings contained herein.

As used herein, the term “long-acting local anesthetic” means an agent that provides analgesia for, typically, 8-16 hours after a peripheral nerve block.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “condition” refers to any physiological status experienced by a subject. The condition may or may not be pathological. Examples of conditions include but are not limited to advanced age, neonatal status, pediatric status, impaired pulmonary function, impaired liver function, impaired cardiovascular function, impaired metabolic function, impaired renal function, impaired gastrointestinal function, impaired neuromuscular function, pregnancy, diabetes, impaired cognitive function, and impaired tolerance of anesthetic or analgesic agents (e.g., due to genetic predisposition, due to metabolic condition).

As used herein, the term “peripheral nerve block” refers to the introduction of an agent near or in a peripheral nerve for the reduction of pain or to provide numbness. Types of peripheral nerve blocks include but are not limited to motor, sensory, differential, and autonomic blocks, and additionally, include but are not limited to brachial plexus (axillary, interscalene, supraclavicular, infraclavicular), individual upper extremity nerve blocks (median, radial, ulnar, musculocutaneous, axillary), sciatic, ankle, metatarsal, oral, femoral, popliteal fossa, saphenous, distal, digital, deep peroneal, superficial peroneal, tibial, sural, and saphenous blocks.

As used herein, “prolonged nerve block” means prolonged pain relief or numbness. The duration of the pain relief or numbness may be 60-90 minutes, 90-120 minutes, 120-150 minutes, 150-180 minutes, 3-6 hours, 6-9 hours, 9-12 hours, 12-14 hours, 14-16 hours, 16-18 hours, 18-24 hours, 24-30 hours, 30-36 hours, 36 hours or more. In certain embodiments, the duration of pain relief or numbness is 24 hours or more. In another embodiment, the duration of pain relief or numbness is three to five days. In a further embodiment, the duration of pain relief or numbness is up to 10 days. Thus, in certain embodiments the duration of pain relief or numbness can from 1, 2, 3, 4, 5, 6, 7, 8, 9 to 10 days.

As used herein, the tem' “pain” means a physiologic and/or psychologic reaction or response to potential or actual stimulus that may result in tissue damage, injury, disease, or other condition(s). Types of pain include but are not limited to acute pain, chronic pain, thermal pain, traumatic pain, chemical pain, inflammatory pain, ischemic pain, blunt pain, sharp pain, prickling pain, visceral pain, and neuropathic pain.

As used herein, the term “antinociception” means reduction in sensitivity to or perception of painful stimuli, without limitation to the type or nature of pain or the manner in which it is imparted.

As used herein, the term “nerve injury” includes but is not limited to direct needle trauma, compression, hematoma, local anesthetic toxicity, surgical trauma, and stretch injury.

As used herein, the term “muscle damage” includes but is not limited to direct needle trauma, compression, hematoma, local anesthetic toxicity, surgical trauma, and stretch injury.

### Kits

Any of the compositions of the present invention, alone or in combination with other compositions of the present invention, may be provided in the form of a kit. The kit may include any and all components necessary or sufficient for administration of peripheral nerve block including, but not limited to, the compositions themselves, syringes, stimulating needles, antiseptic wipes or solutions, sedative agent(s), labels, written and/or pictorial instructions and product information, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. In one embodiment, the kit comprises separate containers housing therapeutically effective amount of each of dexamethasone, epinephrine and bupivacaine or ropivacaine.

### Pharmaceutical Compositions and Routes of Administration

The anesthetic formulation of the present invention can be adapted for use to treat any disease, infection or condition associated with pain, and other indications for which antinociception is desired, alone or in combination with other therapies.

Thus, embodiments of the present invention feature a pharmaceutical composition comprising one or more analgesic and/or anesthetic agent(s) of the invention in an acceptable carrier, such as a stabilizer, buffer, and the like. The compositions of the invention can be administered and introduced into a subject by any standard means, with or without stabilizers, buffers, and the like, to form a pharmaceutical composition. The compositions of the present invention can also be formulated and used as sterile solutions, suspensions for injectable administration, suspensions for continuous infusion, and the other compositions known in the art.

A pharmacological composition or formulation refers to a composition or formulation in a form suitable for administration into a subject or proximal to at least one nerve of a subject, including for example wherein the subject is a human. Suitable forms, in part, depend upon the use or the route of entry. Examples of routes of entry include but are not limited to injection (including but not limited to subcutaneous injection), single injection, serial injection, indwelling catheter or continuous infusion. Such routes of entry should not prevent the composition or formulation from reaching a target cell (i.e., a neuron). For example, injectable pharmacological compositions should be soluble. Other factors are known in the art, and include considerations such as toxicity and forms that prevent the composition or formulation from exerting its effect.

Embodiments of the present invention also include compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). For example, preservatives and stabilizers can be provided. These include sodium benzoate, sorbic acid and esters of p-hydroxybenzoic acid. In addition, antioxidants and suspending agents can be used.

A “therapeutically effective amount” also means a “pharmaceutically effective dose” and is that dose required to prevent, inhibit the occurrence, or treat (alleviate a symptom to some extent, preferably all of the symptoms), e.g. pain. One skilled in the art appreciates that compositions and methods of the present invention find use in multiple types of pain, and that the effective dose may be different for different types of pain. Types of pain include but are not limited to thermal pain, chemical pain, inflammatory pain, ischemic pain, traumatic pain, blunt pain, sharp pain, prickling pain, and visceral pain. The pharmaceutically effective dose depends on the type of condition (e.g., pain), the composition used, the route of administration, the type of mammal being treated, the physical characteristics of the specific mammal under consideration (including but not limited to age, physical condition, surgical or other medical procedures being performed, circulatory capacity, cardiovascular function, pain tolerance, nerve function, liver function), concurrent medication, and other factors that those skilled in the medical arts will recognize.

Compositions of the present invention can also be administered to a subject in combination with other therapeutic compounds to increase the overall therapeutic effect. The use of multiple compounds to treat an indication can increase the beneficial effects while reducing the presence of side effects.

### Examples

The disclosure is further illustrated by the following examples, which are not to be construed as limiting this disclosure in scope or spirit to the specific procedures herein described. It is to be understood that resort may be had to various other embodiments, modifications, and equivalents thereof which may suggest themselves to those skilled in the art without departing from the spirit of the present disclosure and/or scope of the appended claims.

### Example 1

Preparation of Nerve Block Solution for Injection

A 46 cc single dose solution comprising commercially available bupivacaine, epinephrine and dexamethasone sodium phosphate was prepared for injection. The amount of bupivacaine was 3.04 mg/cc, the amount of epinephrine was 4.35 mcg/cc and the amount of dexamethasone sodium phosphate was 0.18 mg/cc.

### Example 2

### Administration of Nerve Block Solution and Results

Informed consent from the patient was obtained and the patient was attached to monitors. The surgical block site was cleaned with alcohol then chlorhexidine for 10 minutes, then prepped with iodine solution. At the bed side, the doctor mixed 14 cc 0.5% Bupivacaine HCL injection+0.1 cc of 1 mg/cc epinephrine injection+1 cc of 4 mg/cc dexamethasone sodium phosphate injection+8 cc of 0.9% bacteriostatic sodium chloride injection to a total volume of 23 cc in a 30 cc syringe. Two such mixtures were prepared.

The patient was sedated with 75 mcg of Fentanyl plus 1.5 mg of Versed. The injection site was injected with 1% lidocaine+EPI 1:200k 1 cc S.Q. A nerve stimulator block needle or an ultrasound block needle advanced percutaneously towards the target nerve. When the needle was close to the target nerve, all 46 cc of the nerve block solution was administered slowly and intermittently.

Co-administration of bupivacaine, epinephrine and dexamethasone sodium phosphate enhanced the duration of sensory blockade when compared with any component alone. The duration of nerve block observed was three to five days in some patients, with other patients advantageously experiencing up to 10 days of pain relief.

The invention is further described in the following numbered paragraphs:

1. An anesthetic formulation, comprising a therapeutically effective amount of each of dexamethasone, epinephrine and bupivacaine or ropivacaine, or a pharmaceutically acceptable salt thereof, as the only analgesic agents and a pharmaceutically acceptable carrier.

2. The anesthetic formulation according to paragraph 1, wherein said therapeutically effective amount of dexamethasone is 0.18 to 0.25 mg/cc.

3. The anesthetic formulation according to paragraph 1, wherein said therapeutically effective amount of epinephrine is at a 1:150,000 to 1:250,000 concentration.

4. The anesthetic formulation according to paragraph 1, wherein said therapeutically effective amount of bupivacaine or ropivacaine is 0.30 to 0.35% total volume of said formulation.

5. The anesthetic formulation according to paragraph 4, wherein said total volume is 42 to 50 cc.

6. The anesthetic formulation according to paragraph 1, having a duration of pain relief of three to five days.

7. The anesthetic formulation according to paragraph 1, having a duration of pain relief of up to 10 days.

8. The anesthetic formulation according to paragraph 1, wherein said therapeutically effective amount of bupivacaine is 3.04 mg/cc, said therapeutically effective amount of epinephrine is 4.35 mcg/cc and said therapeutically effective amount of dexamethasone is 0.18 mg/cc.

9. A method for reducing pain in a subject in need thereof, comprising the step of administering a therapeutically effective amount of each of dexamethasone, epinephrine and bupivacaine or ropivacaine, or a pharmaceutically acceptable salt thereof, as the only analgesic agents as a peripheral nerve block to said patient.

10. The method according to paragraph 9, wherein said subject is human.

11. The method according to paragraph 9, wherein said therapeutically effective amount of dexamethasone is 0.18 to 0.25 mg/cc.

12. The method according to paragraph 9, wherein said therapeutically effective amount of epinephrine is at a 1:150,000 to 1:250,000 concentration.

13. The method according to paragraph 9, wherein said therapeutically effective amount of bupivacaine or ropivacaine is 0.30 to 0.35% total volume.

14. The method according to paragraph 9, wherein said human subject has an additional condition selected from the group consisting of advanced age, neonatal status, pediatric status, impaired pulmonary function, impaired liver function, impaired cardiovascular function, impaired metabolic function, impaired renal function, impaired gastrointestinal function, impaired neuromuscular function, pregnancy, diabetes, impaired cognitive function, and impaired tolerance of anesthetic or analgesic agents.

15 The method according to paragraph 9, wherein the type of said peripheral nerve block is selected from the group consisting of a motor nerve block, a sensory nerve block, a differential nerve block, an autonomic nerve block, a brachial plexus nerve block (axillary, interscalene, supraclavicular, infraclavicular), an individual upper extremity nerve block (median, radial, ulnar, musculocutaneous, axillary), a sciatic nerve block, an ankle nerve block, a metatarsal nerve block, an oral nerve block, a femoral nerve block, a popliteal fossa nerve block, a saphenous nerve block, a distal nerve block, a digital nerve block, a deep peroneal nerve block, a superficial peroneal nerve block, a tibial nerve block, a sural nerve block, and a saphenous nerve block.

16. The method according to paragraph 9, wherein therapeutically effective amount of each of dexamethasone, epinephrine and bupivacaine or ropivacaine is administered by single injection, serial injections, indwelling catheter or continuous infusion.

17. The method according to paragraph 9, wherein the type of said pain is selected from the group consisting of acute pain, chronic pain, thermal pain, traumatic pain, chemical pain, inflammatory pain, ischemic pain, blunt pain, sharp pain, prickling pain, visceral pain, and neuropathic pain.

18. The method according to paragraph 9, wherein said method provides for a duration of pain relief of three to five days.

19. The method according to paragraph 9, wherein said method provides for a duration of pain relief of up to 10 days.

20. A kit, comprising a therapeutically effective amount of each of dexamethasone, epinephrine and bupivacaine or ropivacaine, or a pharmaceutically acceptable salt thereof.

21. The kit according to paragraph 20, further comprising additional components selected from the group consisting of syringes, needles, stimulating needles, catheters, antiseptic wipes or solutions, sedative agent(s), labels, written and/or pictorial instructions and product information, and package environmental controls.

It is to be understood that the invention is not limited to the particular embodiments of the invention described above, as variations of the particular embodiments may be made and still fall within the scope of the appended claims.

1. An anesthetic formulation, comprising a therapeutically effective amount of each of dexamethasone, epinephrine and bupivacaine, or a pharmaceutically acceptable salt thereof, as the only analgesic agents and a pharmaceutically acceptable carrier.

2. The anesthetic formulation according to claim 1, wherein said therapeutically effective amount of dexamethasone is 0.18 to 0.25 mg/cc.

3. The anesthetic formulation according to claim 1, wherein said therapeutically effective amount of epinephrine is at a 1:150,000 to 1:250,000 concentration.

4. The anesthetic formulation according to claim 1, wherein said therapeutically effective amount of bupivacaine or is 0.30 to 0.35% total volume of said formulation.

5. The anesthetic formulation according to claim 4, wherein said total volume is 42 to 50 cc.

6-7. (canceled)

8. The anesthetic formulation according to claim 1, wherein said therapeutically effective amount of bupivacaine is 3.04 mg/cc, said therapeutically effective amount of epinephrine is 4.35 mcg/cc and said therapeutically effective amount of dexamethasone is 0.18 mg/cc.

9-21. (canceled)
